(19) United States
(12) Patent Application Publication
(10) Pub. No.: US 2020/0211676 A1
(43) Pub. Date: Jul. 2, 2020

(54) SYSTEM AND METHOD FOR STORING AND ANALYZING MOLECULAR SEQUENCE DATA

(71) Applicant: Rajesh Perianayagam, Carmel, IN (US)

(72) Inventor: Rajesh Perianayagam, Carmel, IN (US)

(21) Appl. No.: 16/596,030

(22) Filed: Oct. 8, 2019

Related U.S. Application Data
(60) Provisional application No. 62/742,632, filed on Oct. 8, 2018.

(51) Int. Cl. G16B 35/20; G06F 16/25; G06F 16/248; G06F 16/2458; G16B 40/00

(52) U.S. Cl. CPC G16B 35/20; G06F 16/25; G06N 20/00; G06F 16/2458; G16B 40/00; G06F 16/248

(57) ABSTRACT

A database having multiple data sets of molecular sequences and a system for searching the multiple data sets based on user access rights is provided along with a method for increasing the efficiency of future searches of the database.

### RELATED APPLICATION

This application claims priority to U.S. patent provisional application Ser. No. 62/742,632, filed Oct. 8, 2018, to Rajesh Perianayagam, and titled “System and Method for Storing and Analyzing Molecular Sequence Data,” the disclosure of which is hereby incorporate by reference.

### FIELD OF DISCLOSURE

This disclosure relates to a web-based software system and method for storing, analyzing, and managing molecular sequence data within a computerized database.

### BACKGROUND AND SUMMARY OF THE DISCLOSURE

The present disclosure relates to a web-based software system and method for storing, analyzing, and managing public and proprietary molecular sequence data. Databases, such as genome, gene, and protein are useful for scientists. These databases generally contain a large amount of data, and the way to efficiently manipulate these data call attentions of the scientists.

According to one embodiment of the present disclosure, a method of searching molecular sequences is provided comprising the steps of providing at least one database storing a plurality of molecular sequences and at least one input interface having at least one input field, receiving a first search parameter in the at least one input field, performing a first search of the at least one database for molecular sequences matching the first search parameter using a search methodology, outputting a first search result resulting from performing the first search, adjusting the search methodology in response to performing the first search, receiving a second search parameter in the at least one input field, performing a second search of the at least one database for molecular sequences matching the second search parameter using the adjusted search methodology, and outputting a second result resulting from performing the second search.

According to another embodiment of the present disclosure, a method of operating a molecular sequence database is provided comprising the steps of providing at least one database having a plurality a plurality of data sets having molecular sequences and at least one input interface having at least one input field, receiving a first search parameter in the at least one input field, performing a first search of a first proprietary data set of the plurality of data sets for molecular sequences matching the first search parameter, outputting a first search result resulting from performing the first search, receiving a second search parameter in the at least one input field, performing a second search of a second proprietary data set of the plurality of data sets for molecular sequences matching the second search parameter, and outputting a second result resulting from performing the second search.

According to another embodiment of the present disclosure, a method of operating a molecular sequence database is provided comprising the steps of providing at least one database having a plurality a plurality of data sets having molecular sequences and at least one input interface having at least one input field, receiving a first search parameter in the at least one input field, performing a first search of a first proprietary data set of the plurality of data sets for molecular sequences matching the first search parameter, outputting a first search result resulting from performing the first search, receiving a second search parameter in the at least one input field, performing a second search of a second public data set of the plurality of data sets for molecular sequences matching the second search parameter, and outputting a second result resulting from performing the second search.

Additional features of the present disclosure will become apparent to those skilled in the art upon consideration of the following detailed description of the illustrative embodiment exemplifying the best mode of carrying out the disclosure as presently perceived.

### BRIEF DESCRIPTION OF THE DRAWINGS

The aforementioned aspects of this disclosure will grow to be appreciated at a greater level once references to the following accompanying illustrations are expounded upon.

FIG. 1 is a method for gathering, storing, analyzing, and managing molecular sequence data within a database.

FIG. 2a is a diagrammatic view of the database separated into a plurality of data sets.

FIG. 2b is a diagrammatic view of a system for users to access, download, and store molecular sequence data on the database on a server.

FIG. 3 is an example of a system for users to access and store molecular sequence data on this database through cloud computer services.

FIG. 4A is a diagrammatic view of a user's dashboard.

FIGS. 4B and 4C are partial enlarged diagrammatic views of portions of FIG. 4A.

FIG. 5A is a diagrammatic view of a user's search for specific genomes and results of the search.

FIGS. 5B and 5C are partial enlarged diagrammatic views of portions of FIG. 5A.

FIGS. 6A and 6B are diagrammatic views of a user's search for a specific gene and the results of the search.

FIGS. 7A and 7B are diagrammatic views of a user's search for a specific protein and the results of the search.

FIGS. 8A and 8B are diagrammatic views of a user completing a search to conduct a BLAST analysis and the results of the search.

FIG. 9 is a diagrammatic view of a graphical overview of a BLAST analysis.

FIGS. 10A-10C are diagrammatic views of a user browsing a genome.

FIG. 11 is a diagrammatic view of the system architecture.

Wherein, illustrations depicted are manifestations of the disclosure, such illustrations shall in no way be interpreted as limiting the scope of the disclosure.

For the purposes of promoting and understanding of the principals of the disclosure, reference will now be made to the embodiments illustrated in the drawings, which are described below. The embodiments disclosed below are not intended to be exhaustive or limit the disclosure to the precise form disclosed in the following detailed description. Rather, the embodiments are chosen and described so that others skilled in the art may utilize their teachings. It will be understood that no limitation of the scope of the disclosure is thereby intended. The disclosure includes any alterations and further modifications in the illustrative devices and described methods and further applications of the principles of the disclosure which would normally occur to one skilled in the art to which the disclosure relates.

### DETAILED DESCRIPTION

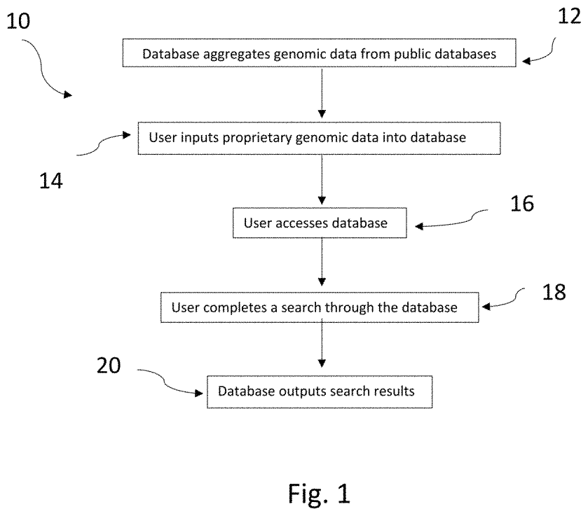

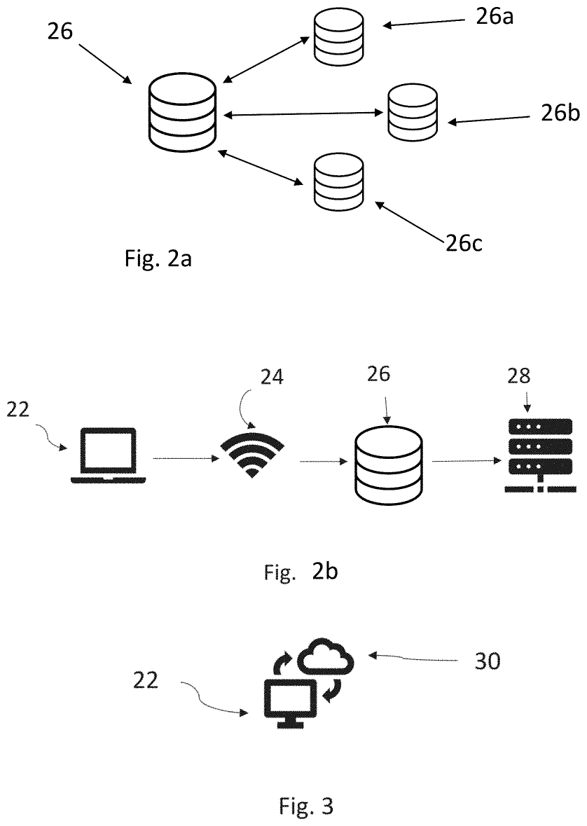

Referring to FIG. 1, a schematic of a method 10 for storing and analyzing molecular sequence data using web-based software is shown. Initially in a data aggregation step 12, a web-based database 26 (as shown in FIG. 2) gathers and organizes sequence data from public databases (not shown), such as but not limited to, the National Center for Biotechnology Information. This data is updated regularly as new data is added to these public databases. Database 26 integrates the data from the public databases. The user can perform an input step 14 to input proprietary data to create a more comprehensive database 26 for the user's needs.

Only clients who input this proprietary data into database 26 can view the proprietary data. As shown in FIG. 2a, database 26 is divided into a plurality of data sets 26a, 26b, 26c. According to the present disclosure, at least one of the data sets 26a is included data from public databases and other data sets 26b, 26c include proprietary data provided by users. For example, data set 26b may include data provided by a first user, such as a first university or company and data set 26c may include data provided by a second user, such as a second university or company.

Users can access this database 26 in an access step 16 in different ways. As shown in FIG. 2b, a user can utilize a personal computer 22, with the use of the internet 24 to perform access step 16 and download portions of database 26 to a user's internal servers 28 and/or the database may be stored on an external server 28. As shown in FIG. 3, a user can utilize a personal computer 22 to access portions of database 26 through cloud computer services 30, such as but not limited to, Amazon-brand Web Services or Google-brand Cloud Platform. These examples allow a user to access portions of database 26 remotely from wherever, and whenever, a user has internet connection.

Each user has a user id and password to access portions of database 26 and may share that password if the user wants another person to view the user's proprietary data. Access to each data set is controlled by the user id. For example, a first user may have access to public data set 26a and their own proprietary dataset 26b, but does not have access to data set 26c that contains the proprietary data of a second user. A second user may have access to public data set 26a and their own proprietary data set 26c, but does not have access to data set 26b that contains the proprietary data of the first user.

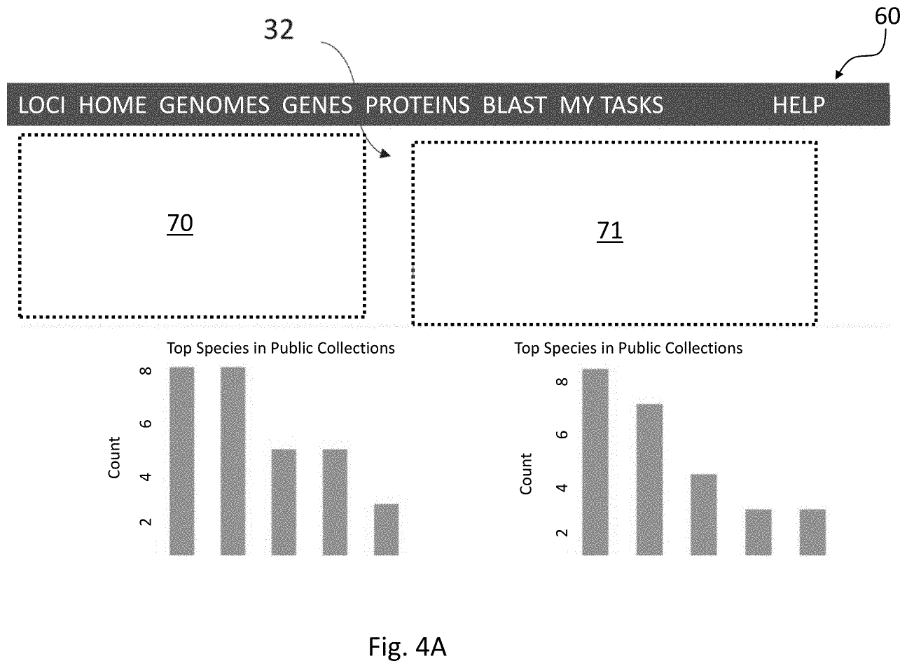

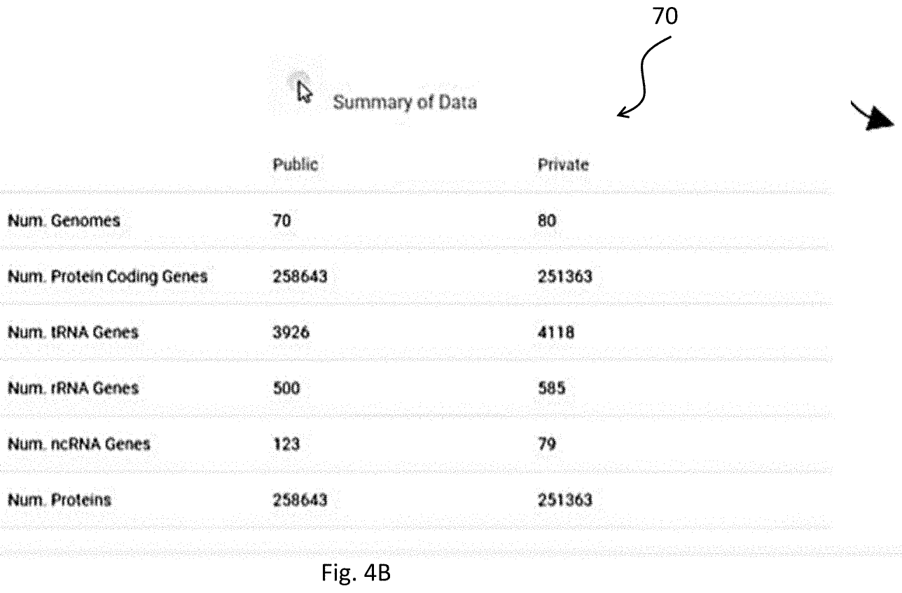

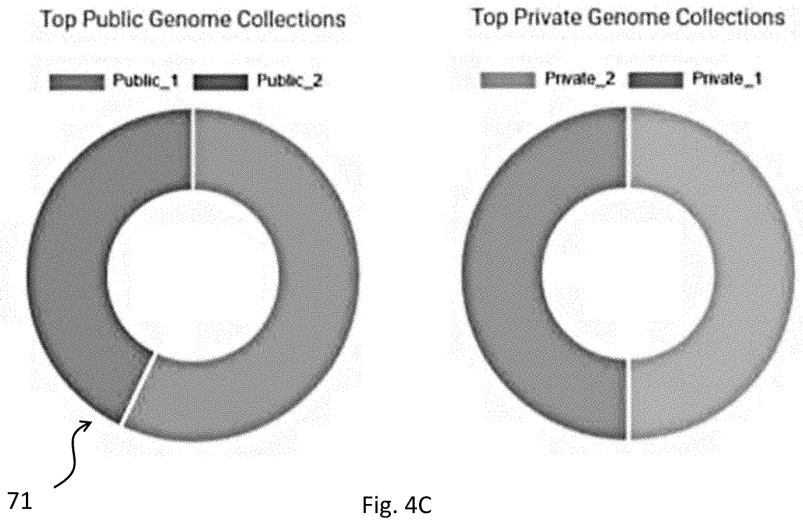

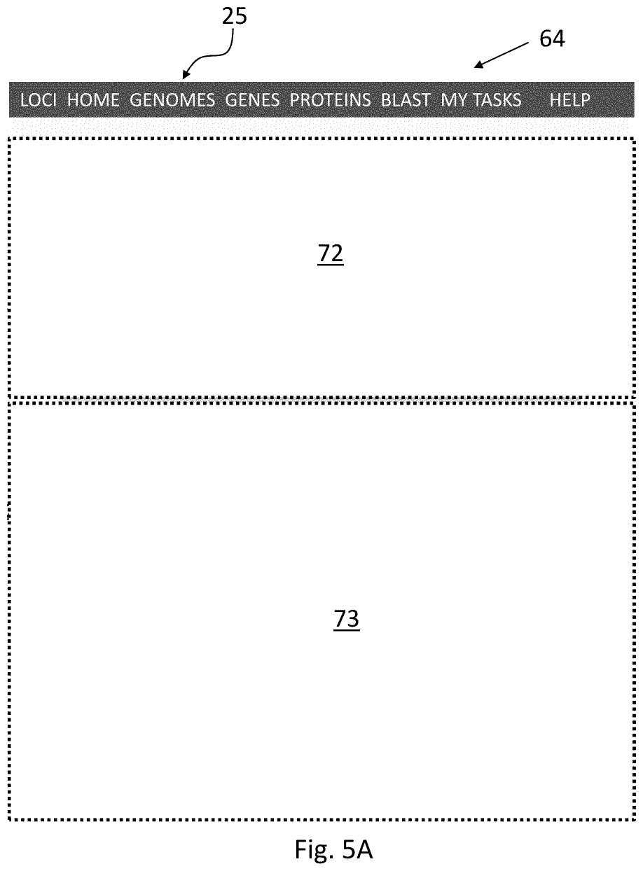

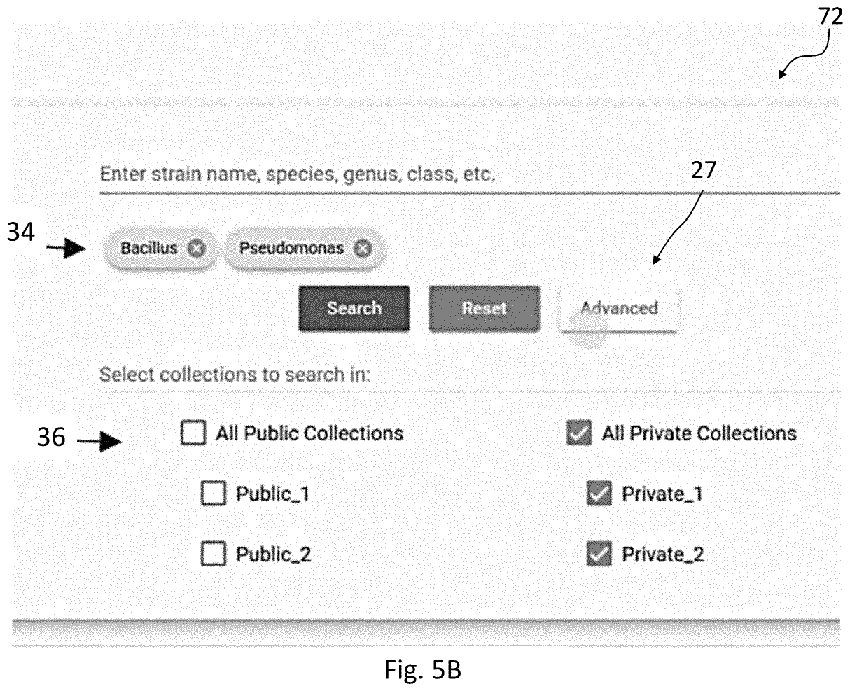

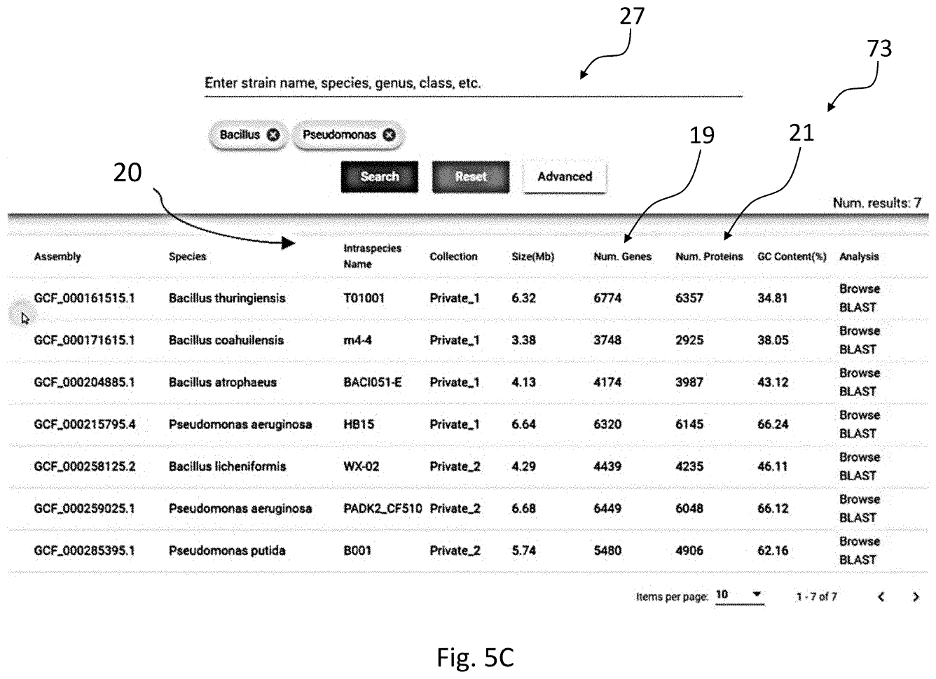

As shown in FIGS. 4A-4C, in which the portions 70, 71 of FIG. 4A are respectively shown in FIGS. 4B and 4C, a user's dashboard 32 includes a summary and graphical representations of data collected from public and private data collections of a specific user stored in their own data set 26b, 26c. The summary can include, but is not limited to, the number of genomes, number of protein coding genes, number of tRNA genes, number of rRNA genes, number of ncRNA genes, and number of proteins within each collection. Each of the genomes, protein coding genes, tRNA genes, rRNA genes, ncRNA genes, and proteins data within each collection can be a data entry. Each data entry 21 (as shown in FIGS. 5A-5C) has multiple indexes 23 each indicating a characteristic of the data entry, like the entry's collection, type, ID, species, assembly, length, etc. In search step 18, a user can search for specific molecular sequences based on the use of various keywords, such as but not limited to, certain taxonomic ranks and genes, proteins, and other macromolecules or macromolecular domains. A user can further limit a search to specific public databases or private databases. Database 26 outputs a result 20 based on the user's search from search step 18.

The instant system has a user input interface 64 having a first input field 25, which receives a first command of search (like, for example, a click of selections). The first command of search, for example, may indicate the categories of interest, including the options of genomes, genes, and proteins. Accordingly, user interface 64 of the system can provide a second input field 27. The second input field 27 is provided according to the selection made by the first command of search. The second input field 27 is configured to receive at least one second search parameter/command of search. For example, the second command of a search can be the name of genes or genomes, ID, species, and class, etc. These second commands of search can be aggregated as shown in the specific genomes 34 in FIG. 5B. Based on the second command, the system can match the command (such as a string of texts) to the existing the name of genes or genomes, ID, species, and class, etc in the databases. Thus, the system can provide the user a list of matched pre-determined options (like the genomes 34), and the user can select on of the options by at least one first selection command. Then, the system can use the at least one chosen selection (like the genomes 34) to perform a search of the data entries based on the at least one first selection command. By this multi-layer commands sequence, the search engine of the computer system can perform a more efficient search by excluding the date entries that are not within the categories of interest. The search result 20 (in FIG. 5C) can be shown in a table 19.

Additionally, the system can receive second election command of the pre-determined options to select among a plurality of data collections. For example, the selection can designate the targeted database incorporated in the whole system as shown by public and/or private data collections 36 in FIG. 5B.

As shown in FIGS. 5A-5C, in which the portions 72, 73 of FIG. 5A are respectively shown in FIGS. 5B and 5C, a user can complete search step 18 for specific genomes 34 within either a public and/or private data collection 36. Search results 20 for a genome 34 include, but are not limited to, assembly, species, interspecies name, collection type, size (Mb), number of genes, number of proteins, GC content (%), and an analysis column to either browse the genome or to conduct a Basic Local Alignment Search Tool (BLAST). Specifically, FIGS. 5A to 5C are an example of a user completing search step 18 to find out how many Bacillus and Pseudomonas genomes are present in their proprietary microbial collections and/or the public collections.

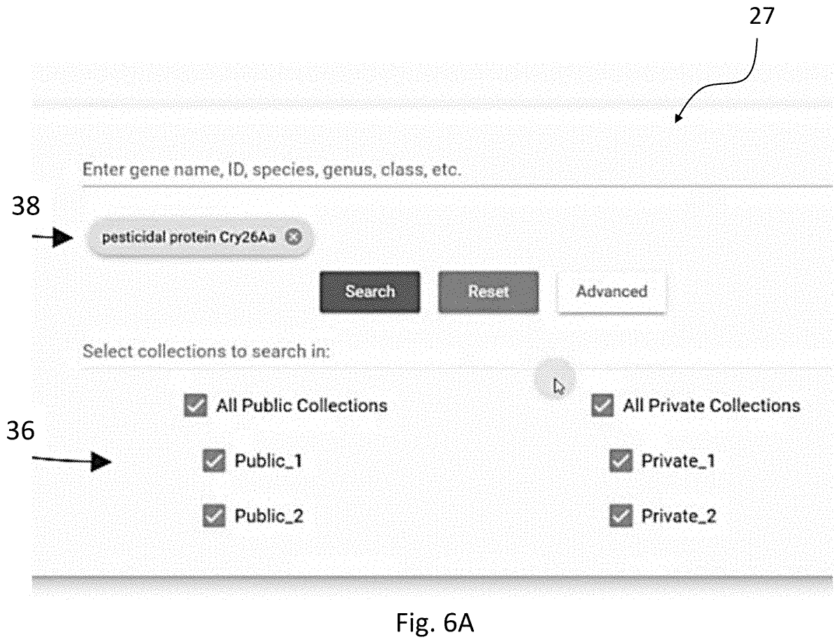

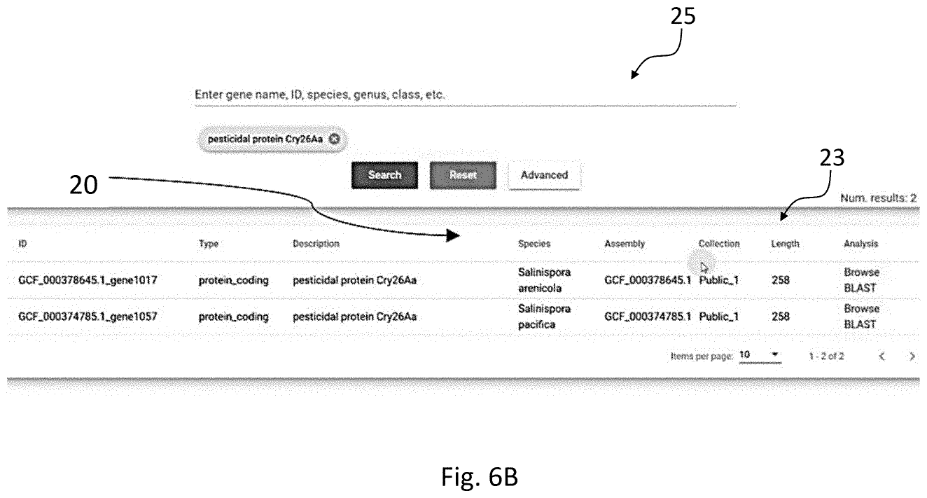

As shown in FIGS. 6A and 6B, a user can complete search step 18 for a specific gene 38 within either a public and/or private data collection 36. Search results 20 for a gene 38 include, but are not limited to, ID, type, description, species, assembly, collection type, strain length, and an analysis column to either browse the strains that contain this gene or to conduct a BLAST analysis. Specifically, FIGS. 6A and 6B are an example of a user completing search step 18 to discover a list of Cry26Aa gene carrying strains in their proprietary microbial collections that the user can choose from for bio-insecticidal testing.

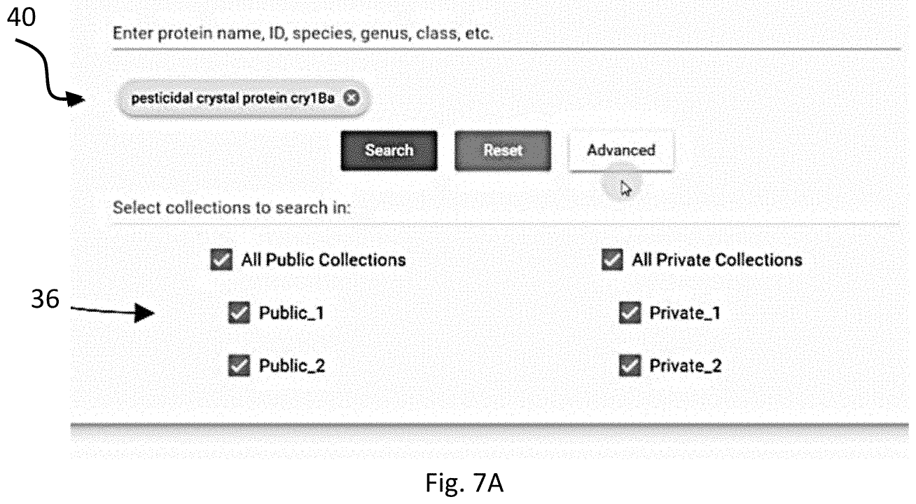

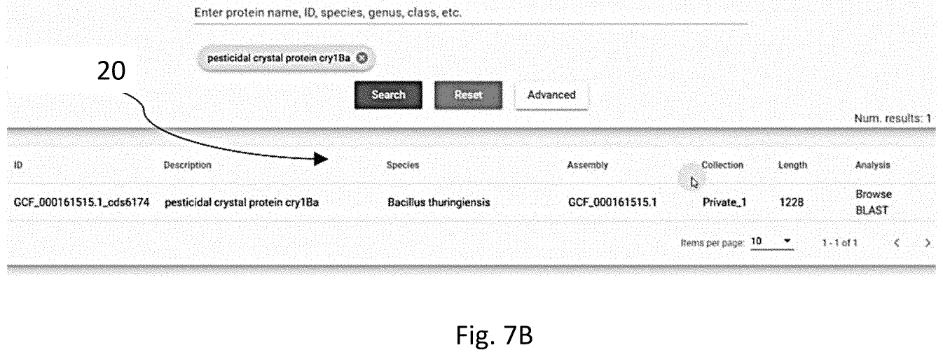

As shown in FIGS. 7A to 7B, a user can complete search step 18 for a specific protein 40 within either a public and/or private data collection 36. Search results 20 for a protein include, but are not limited to, ID, description, species, assembly, collection type, strain length, and an analysis column to either browse the strains that contain this gene or to conduct a BLAST analysis. Specifically, FIGS. 7A to 7B are an example of a user completing search step 18 to discover the list of Cry1Ba protein carrying strains in their proprietary microbial collections that the user can choose from for bio-insecticidal testing.

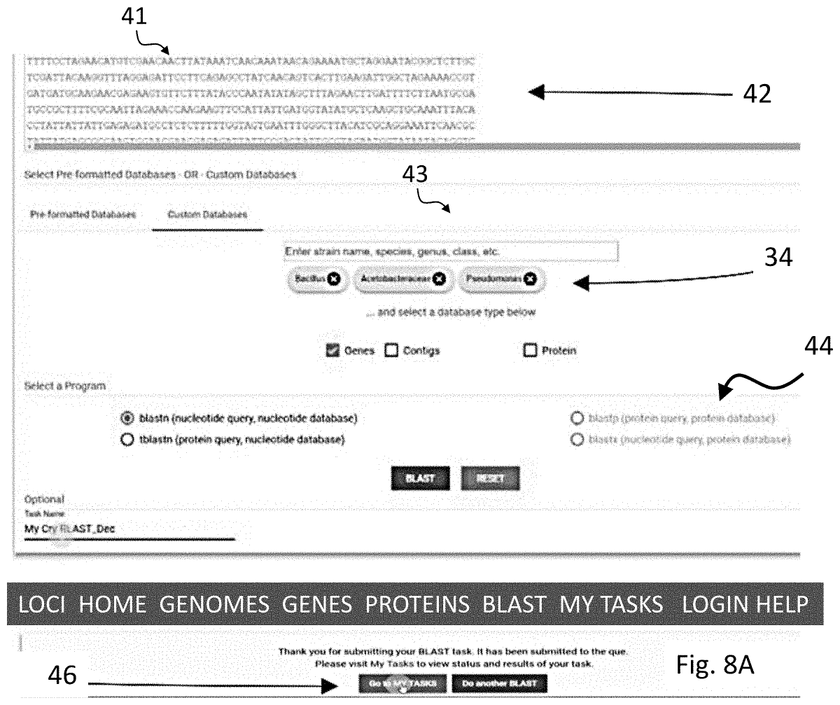

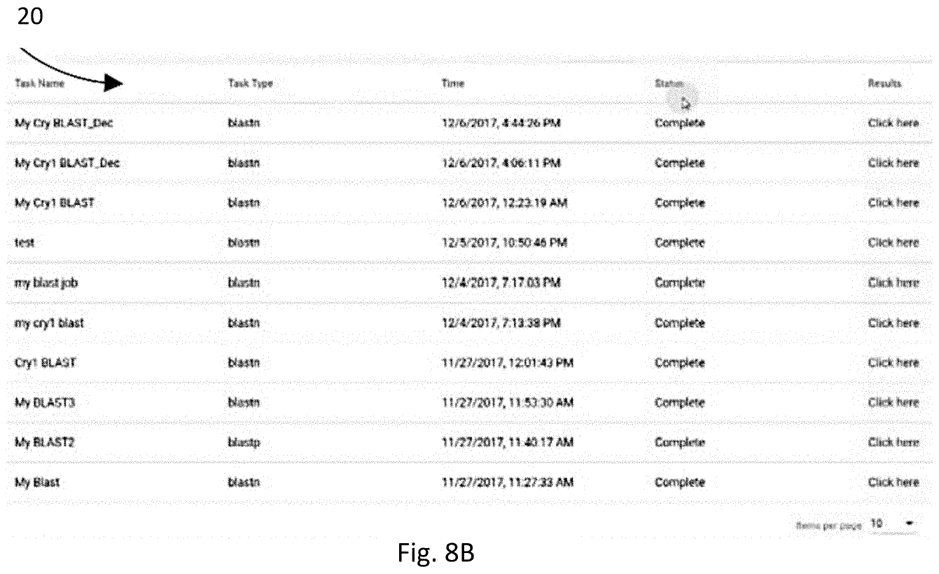

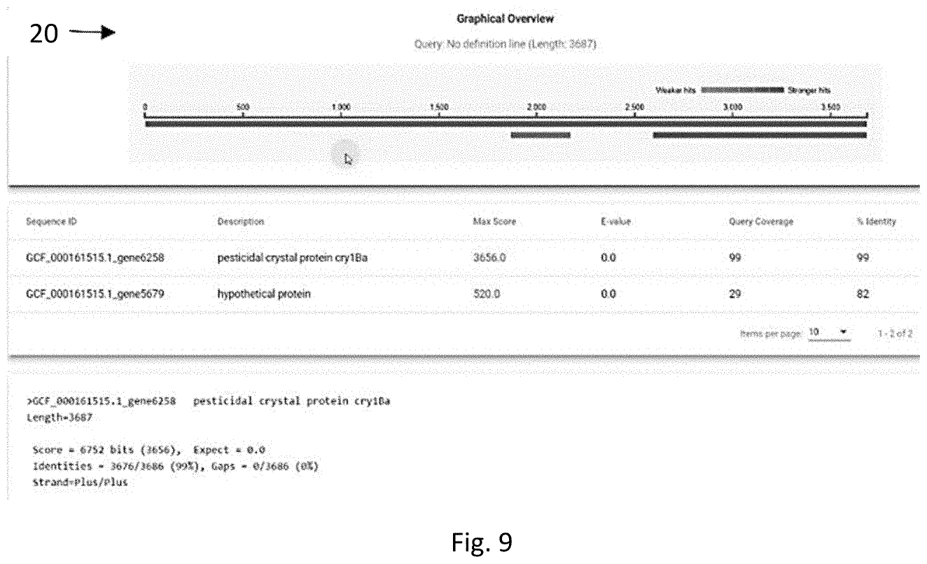

As shown in FIGS. 8A to 8B, a user can input a specific molecular sequence 42 in the input field and complete search step 18 for a specific genome 34 that has a significant similarity to the user's input value(s). In an embodiment, genome 34 in 26 database each has an individual sequence code. In an embodiment, a second input field 43 can receive a second input command, like a string of text indicating the type of genome the user is interested in. The system can provide a list of matched pre-determined options (like genome 34), and the user can provide at least one selection command (like clicking of his/her mouse) to select the pre-determined options of interest. A user can pick a specific type of BLAST program 44 for a search, such as but not limited to, a blastn, tblastn, blastp, or blastq search. Search results 20 for a BLAST search step 18 are stored in the user's tasks 46. FIG. 9 displays a user's BLAST analysis results 48 which include, but are not limited to, a sequence ID, strain description, max score of the strained based on the number of hits between the input sequence 42 and the output strain, E-value, query coverage, and % of identity. Specifically, FIGS. 8A to 8B and FIG. 9 are an example of a user completing a search step 18 to perform a custom BLAST analysis to discover statistically significant similarity of Cry1Ba nucleotide sequences in a desired subset of genomes from their microbial genome databases. This system may perform a better analysis function by the above pre-selection action to exclude the data entries out of the user's interest. This system may further provide an input field 41, which can receive and interpret the sequence code.

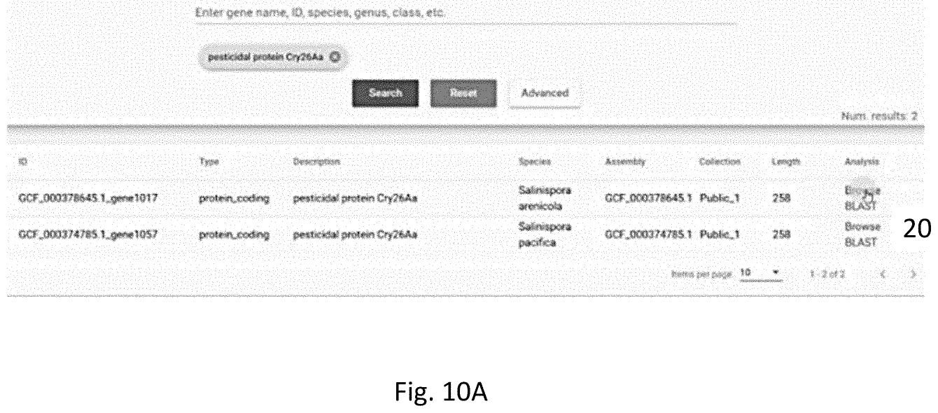

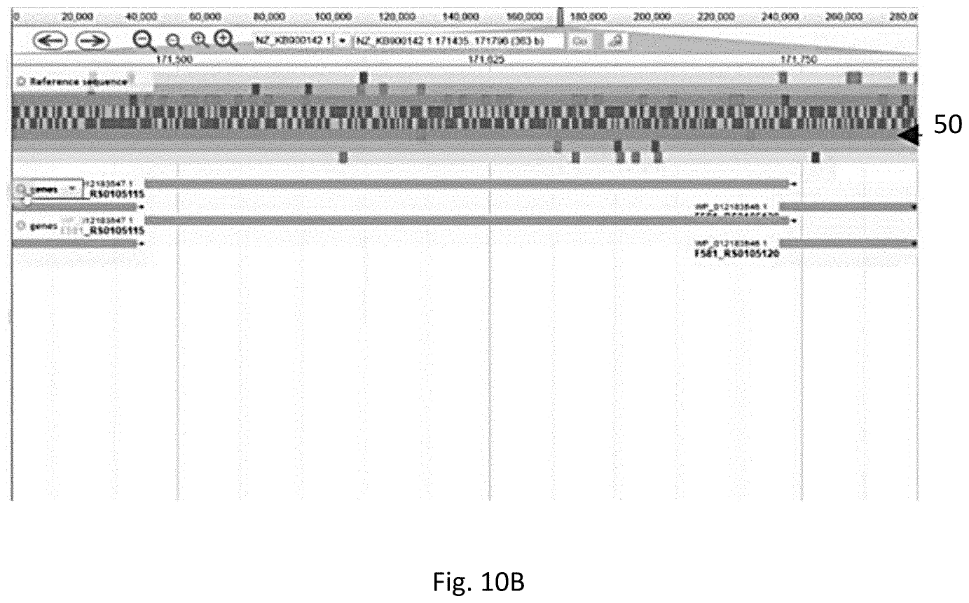

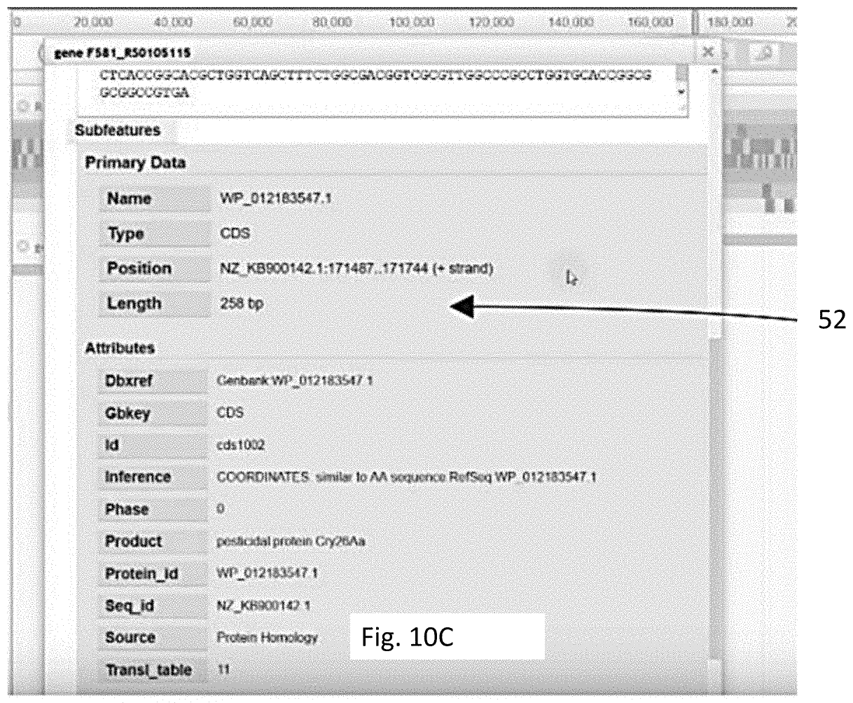

As shown in FIGS. 10A to 10C, once a user receives search results 20, the user can view a visualization 50 of the reference and output molecular sequences by “browsing” the result. This visualization 50 also provides the user with features of the sequences, such as but not limited to, name, type, position, length, ID, protein, phase, source, and inference in an expanding menu 52. Specifically, FIGS. 10A to 10C are an example of a user trying to understand the neighborhood of newly discovered insecticidal gene Cry26Aa in a bacterial strain, Bacillus, for a purpose of finding other potential insecticidal proteins for bio-insecticidal testing.

Search results 20 provide a comparison between the user's search input value(s) and the specific molecular sequences that are found within database 26. This comparison can include, but is not limited to, the length of molecular sequences, a description of the species, and a percent value representing how similar the input of search step 18 and output sequences are. These results 20 include the ability for the user to conduct BLAST analyses based on a specific return located within search result 20 and the user's search input during search step 18. Database 26 also provides integrated software for interactive visualization of the data.

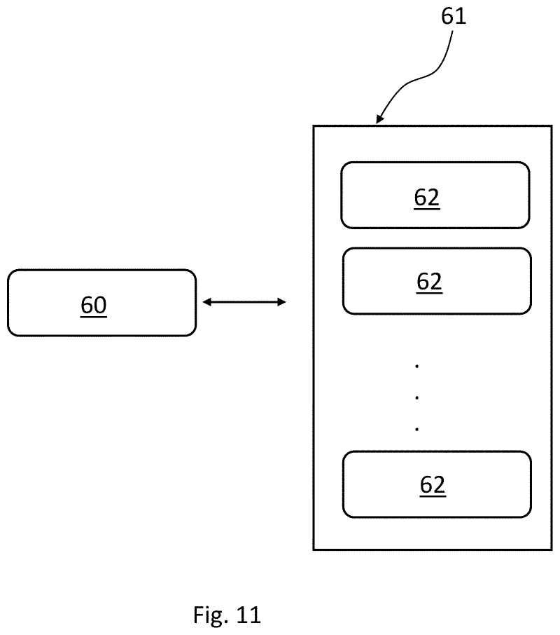

Additionally, as shown in FIG. 11, the instant system includes a frontend platform 60 (like webpages, end-user devices as shown in FIG. 4) and a least one backend module 62 on a computer 61, like a server. Frontend platform 60 provides flexible column headers on Genomes/Genes/Proteins/BLAST results (for example, in the table 19 of FIG. 5C). The format of the result can be set by the user. Frontend platform 60 further provides query builder for various ways including many operators, such as “And”, “Or”, “Not,” for “search” to conduct a precise search. Frontend platform 60 further allows user to download the results based on desired % similarity after the alignment analysis (like the BLAST analysis). Frontend platform 60 also incorporates hyperlink to unique IDs, which is connected to the summary page that contains all entered information about unique IDs. Frontend platform 60 also allows a user to uploading all or any type of omics data (or other type of bioinformatic data) from the user's desktop into the server or the database. Frontend platform 60 also provides pop-up definitions of titles when the user's cursor is hovered around them. Frontend platform 60 also connects all unique IDs to a “Browse” hyperlink for visualization. Frontend platform 60 also highlights duplicate entries in the search result. These measures allow the user to navigate the complicated bioinformatic data on a computer to facilitate the easier operation of the computer.

Modules 62 may include: a metagenomics module for storing, managing, mining, analyzing and visualizing metagenomics data; a pedigree module using any type of omics data to discover the genetic relationship among microbes, plants and other organisms; database for all -omics data types (genomics, metagenomics, metatranscriptomics, transcriptomics, proteomics, 16s rRNA and metabolomics); a genomic Intelligence module to perform omics data mining for data access and gain insights using real time data through natural language-based questions and to perform all functionalities of our technology using natural language-based voice commands; and an artificial Intelligence module using machine learning algorithms for predictive analytics combining all omics data or any -omics data to discover beneficial microbes, plants, animals and humans.

With each search, the search methodology improves using machine learning or other forms of artificial intelligence. For example, method 10 may us a first search methodology when performing a first search and outputting the first search results. Using machine learning, method 10 adjusts the search methodology after the first search to improve the search by making it more efficient, accurate, etc. to increase the performance (speed, accuracy, etc.) of the search. Method 10 then using a second search methodology (i.e. the result of adjustments to the first search methodology) when performing a second search and outputting the second search result. Method 10 continues to improve the search methodology with each search. Because multiple users are using method 10, the search methodology improves at a faster rate than if a single user was using method 10. Additionally, because method 10 is being used on multiple data sets 26a, 26b, 26c, etc., the search methodology improves at faster rate that if it was being used on a single data set or fewer data sets. Thus, as method 10 is used repeatedly by a single user on their proprietary data set (e.g. 26b) and/or public dataset 26a, the search methodology improves and as multiple users use method 10 on multiple proprietary data sets (e.g. 26b, 26c, etc.) and/or public data set 26a, the search methodology improves. Because the search methodology improves based on experience across multiple users and multiple database and/or data sets, it improves faster than if the search improved based on a single user and a single database and/or data set.

Adjustments of the search methodology carryover from one user (and their respective datasets) to other users (and their respective data sets). For example, improvements to the search methodology based on a first user's search of the first user's respective data sets may next be used in a second user's search of the second user's respective data sets. Thus, each user's search benefits from a previous search regardless of who's search was performed and on which data sets the previous search was performed.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practices in the art to which this disclosure pertains.

What is claimed is:

1. A method of searching molecular sequences comprising the steps of
providing at least one database storing a plurality of molecular sequences and at least one input interface having at least one input field,
receiving a first search parameter in the at least one input field,
performing a first search of the at least one database for molecular sequences matching the first search parameter using a search methodology,
outputting a first search result resulting from performing the first search,
adjusting the search methodology in response to performing the first search,
receiving a second search parameter in the at least one input field,
performing a second search of the at least one database for molecular sequences matching the second search parameter using the adjusted search methodology, and
outputting a second result resulting from performing the second search.

2. The method of claim 1, wherein the database includes a plurality of data sets and the first search is limited to a first subset of the plurality of data sets and the second search is limited to a second subset of the plurality of data sets that is different than the second subset.

3. The method of claim 2, wherein the first data set is proprietary to a first user and the second data set is proprietary to a second user.

4. The method of claim 3, wherein the first search parameter is received from the first user and the second search parameter is received from the second user.

5. The method of claim 1, wherein database includes a plurality of data sets including at least one public data set and at least one proprietary data set.

6. The method of claim 1, wherein the adjusting step results from machine learning.

7. A method of operating a molecular sequence database comprising the steps of
providing at least one database having a plurality a plurality of data sets having molecular sequences and at least one input interface having at least one input field,
receiving a first search parameter in the at least one input field,
performing a first search of a first proprietary data set of the plurality of data sets for molecular sequences matching the first search parameter,
outputting a first search result resulting from performing the first search,
receiving a second search parameter in the at least one input field,
performing a second search of a second proprietary data set of the plurality of data sets for molecular sequences matching the second search parameter, and
outputting a second result resulting from performing the second search.

8. The method of claim 7, wherein the first search is limited to a first subset of the plurality of data sets and the second search is limited to a second subset of the plurality of data sets that is different than the second subset.

9. The method of claim 8, wherein the first data set is proprietary to a first user and the second data set is proprietary to a second user.

10. The method of claim 9, wherein the first search parameter is received from the first user and the second search parameter is received from the second user.

11. The method of claim 7, wherein the plurality of data sets including at least one public data set and at least one proprietary data set.

12. A method of operating a molecular sequence database comprising the steps of
providing at least one database having a plurality a plurality of data sets having molecular sequences and at least one input interface having at least one input field,
receiving a first search parameter in the at least one input field,
performing a first search of a first proprietary data set of the plurality of data sets for molecular sequences matching the first search parameter,
outputting a first search result resulting from performing the first search,
receiving a second search parameter in the at least one input field,
performing a second search of a second public data set of the plurality of data sets for molecular sequences matching the second search parameter, and
outputting a second result resulting from performing the second search.

13. The method of claim 12, wherein the first search is limited to a first subset of the plurality of data sets and the second search is limited to a second subset of the plurality of data sets that is different than the second subset.

14. The method of claim 13, wherein the first search parameter is received from the first user and the second search parameter is received from the second user.

15. The method of claim 12, wherein the plurality of data sets including at least one public data set and at least one proprietary data set.

16. The method of claim 12, wherein the step of performing the first search and the step performing the second search occur substantially simultaneously.
